(19) United States
(12) Patent Application Publication
(10) Pub. No.: US 2013/0267490 A1
(43) Pub. Date: Oct. 10, 2013

(54) TOPICAL PHARMACEUTICAL COMPOSITION COMPRISING NANONIZED SILVER SULFADIAZINE AND CHLORHEXIDINE GLUCONATE

(71) Applicants: Neeta Gupta, Gurgaon (IN); Simrata Bedi, Gurgaon (IN); Jyoti Srivastava, Gurgaon (IN); Vinod Kumar Arora, Gurgaon (IN)

(72) Inventors: Neeta Gupta, Gurgaon (IN); Simrata Bedi, Gurgaon (IN); Jyoti Srivastava, Gurgaon (IN); Vinod Kumar Arora, Gurgaon (IN)

(73) Assignee: RANBAXY LABORATORIES LIMITED, New Delhi, Delhi (IN)

(21) Appl. No.: 13/814,086

(22) PCT Filed: Jul. 21, 2011
(86) PCT No.: PCT/IB2011/053263

§ 371 Date: May 22, 2013

(30) Foreign Application Priority Data
Aug. 2, 2010 (IN) 1821/DEL/2010

(51) Int. Cl. A61K 31/555; A61K 31/155

(52) U.S. Cl. CPC A61K 31/555; A61K 31/155
USPC 514/184

(57) ABSTRACT

The invention relates to an improved topical pharmaceutical composition for burn treatment and microbial infections on human beings or animals. The pharmaceutical composition comprises 0.1% w/w to 1% w/w of an antimicrobial drug, i.e., silver sulfadiazine and 0.2% w/w antiseptic, i.e., chlorhexidine gluconate; wherein silver sulfadiazine is in nanonized form.

### FIELD OF THE INVENTION

The present invention relates to an improved topical pharmaceutical composition of nanonized silver sulfadiazine, and processes for its manufacture.

### BACKGROUND OF THE INVENTION

The invention relates to an improved topical pharmaceutical composition for the treatment of burns and/or microbial infections in human beings or animals. The pharmaceutical composition includes an antimicrobial drug, i.e., silver sulfadiazine, and an antiseptic, i.e., chlorhexidine gluconate, wherein the silver sulfadiazine is in nanonized form.

Silver sulfadiazine was first described in 1943 by Wruble and was found to be mildly antiseptic. U.S. Pat. No. 3,761,590 describing a process for preparing a thick cream ointment containing silver sulfadiazine, rejuvenated the compound for the topical treatment of burns. In its cream form, the 1% w/w concentration of this active drug has been in clinical use in the United States since 1973.

Chlorhexidine is a bisbiguanide antiseptic and disinfectant effective against a wide variety of bacteria, some fungi and some viruses. It is used clinically in various preparations for disinfecting purposes.

The antimicrobial effect of silver sulfadiazine and chlorhexidine compounds has been clinically established. It has been well known that silver sulfadiazine is effective against a wide variety of gram-positive and gram-negative organisms, including Pseudomonas and Candida. The prior art discloses a number of formulations of silver sulfadiazine for treatment of burns.

EP 0 326 145 discloses a composition for the topical treatment of herpes infections, varicella, eczema, and burns (2nd and 3rd degree) comprising 0.01% to 10% silver sulfadiazine, 0.01% to 10% polyhydric alcohols, and optionally a local anesthetic.

BE 892421 relates to stable ointments and lotions for the treatment of mucosal infections without causing irritation, which contain silver sulfadiazine and a hydrophilic excipient.

FR 2424740 describes lotions, ointments, and powders, containing finely-divided silver sulfadiazine or zinc sulfadiazine. These salts were prepared in situ from sodium sulfadiazine. Thus, a lotion was prepared by dissolving sodium sulfadiazine in water, homogenizing with Tween® 80, paraffin, isopropyl palmitate, sorbitan monooleate, Myrj® 52, and stearyl alcohol. The mixture was homogenized with silver nitrate in water at 3000 rpm to form finely-divided silver sulfadiazine with a particle size<5 μm and with 90% of particle size<0.7 μm.

IN 1038/KOL/2005 relates to a therapeutic composition for treating burns and includes as active ingredients the following in amounts shown: (a) sucralfate—1%-8% wt/wt; (b) silver sulfadiazine—0.5%-2% wt/wt; and (c) chlorhexidine gluconate—0.1% -0.3% wt/wt. These are admixed with pharmaceutically acceptable or herbal plant extracts, additives, excipients, adjuvants, fillers, humectants and/or stabilizers. It also discloses a process for preparing the above-mentioned therapeutic composition effective for burn wound management.

U.S. Pat. No. 6,987,133 discloses a topical spray preparation for burn treatment that includes silver sulfadiazine dispersed or solubilized in the cream or lotion base matrix, which can be sprayed directly from a common trigger spray device.

Commercial formulations containing chlorhexidine gluconate 0.2% w/w, and silver sulphadiazine 1% w/w, e.g., Silvazine, Nisburn®, or Silverex® cream, are available in the market; however, the particle size range of the active in these formulations is in the micronized range.

The activity of silver sulfadiazine, in the cream form, may be influenced by the following factors:
a) the release rate of the active ingredient from the cream matrix in the wound environment;
b) the particle size and solubility of the active drug in the fluids of the wound bed; and
c) the stability of the active ingredient in the cream matrix.

Amongst these factors, particle size is one critical parameter which affects the solubility and release of the active ingredient from the pharmaceutical composition at the wound site. A smaller particle size will lead to increased antimicrobial effectiveness. Therefore, further size reduction of silver sulfadiazine particles, as compared to the marketed micronized product, may result in greater antimicrobial effectiveness based on enhanced solubility of silver sulfadiazine.

Particle size reduction to improve the drug performance has long been known and used in the pharmaceutical industry. Nanonization, i.e., particle size reduction to the nano-size range is a known technique for improving the solubility of the active particles, thereby leading to improved absorption and better therapeutic efficacy. However, selection of the optimum particle size range as well as the selection of the method for particle size reduction remains critical for the chosen active ingredient. Nesamony et al, in their article entitled “IPM/DOSS/Water Microemulsions as Reactors for Silver Sulfadiazine Nanocrystal Synthesis”, published in the Journal of Pharmaceutical Sciences, 94:6, June 2005, has disclosed a method of preparing silver sulfadiazine nanocrystals in situ by mixing sodium sulfadiazine and silver nitrate using microemulsion technique. The silver sulfadiazine nanocrystals so prepared have a particle size of ˜670 nm, as measured by a laser diffraction technique. The preparation of nanocrystals using this microemulsion technique is a complex process. Moreover, the article does not disclose any formulation composition details or any studies on efficacy or therapeutic improvement using the silver sulfadiazine nanocrystals.

There still remains a need for a topical pharmaceutical composition that includes silver sulfadiazine in a nano-size range which demonstrates improved efficacy over the already marketed product.

Therefore, the object of the present invention is to provide an improved topical pharmaceutical composition, which includes an antimicrobial drug, i.e., silver sulfadiazine, in a weight ratio of 0.1%-1% w/w and an antiseptic, i.e., chlorhexidine gluconate, in a weight ratio of 0.2% w/w, wherein the silver sulfadiazine is in nanonized form. The pharmaceutical composition of the present invention is advantageous over the currently available micronized products due to improved efficacy, thereby leading to a dose reduction, faster wound healing as a result of quicker absorption of fine sized particles having increased surface area and no significant increase in toxicity as a result of size reduction.

### SUMMARY OF THE INVENTION

In one general aspect, the present invention is related to a topical pharmaceutical composition which includes 0.1%-1% w/w silver sulfadiazine, 0.2% w/w chlorhexidine gluconate, and one or more pharmaceutically acceptable excipients; wherein the silver sulfadiazine has a Z-average molar mass between 150-500 nm.

Embodiments of this aspect of the present invention may include one or more of the following features. For example, the pharmaceutically acceptable excipients may include one or more of viscosity enhancers, emulsifying agents, fragrance/perfumes, preservatives, chelating agents, pH modifiers, and antioxidants.

Suitable viscosity enhancers include one or more of soft paraffin, aluminum stearate, cetostearyl alcohol, propylene glycol, polyethylene glycols, povidone, wool-fat, hydrogenated lanolin, beeswax or mixtures thereof.

Suitable emulsifying agents include one or more of cetomacrogol, non-ethoxylated glyceryl monostearate, carbopols, cetearyl alcohol, sodium stearoyl lactylate, lecithin or mixtures thereof.

Suitable preservatives include one or more of methylparaben, propylparaben, benzyl alcohol, benzoic acid, sodium benzoate, chlorocresol, sorbic acid and its salt, phenylethyl alcohol or mixtures thereof.

Suitable chelating agents include one or more of dimercaprol, ethylene diamine tetra acetic acid (EDTA), ethylene glycol tetra acetic acid, deferoxamine, alfa lipoic acid or mixtures thereof.

Suitable pH modifiers include one or more of citric acid, sodium citrate; acetic acid, sodium acetate; phosphoric acid, disodium orthophosphate; and borax, sodium hydroxide.

Suitable antioxidants include one or more of hydrogen peroxide, sodium metabisulfite, butylated hydroxytoluene, butylated hydroxyanisole, propyl gallate, ethyl gallate, methyl gallate, ascorbic acid, tocopherol, or mixtures thereof. For example, the antioxidant may be a combination of hydrogen peroxide, sodium metabisulfite, and butylated hydroxytoluene.

The composition of the present invention may be in the form of a cream, lotion, ointment or gel.

In another general aspect, the present invention provides for a process for the preparation of the topical pharmaceutical. The process includes the steps of:
a) dispersing the silver sulfadiazine and the viscosity enhancer(s) in water;
b) charging the dispersion of step a) in a Dynomill and unloading the slurry when silver sulfadiazine attains a particle size of Z-average value between 150-500 nm;
c) emulsifying suitable oil and aqueous phases containing excipients;
d) adding chlorhexidine gluconate and the slurry containing nanonized silver sulfadiazine to the emulsion of step c); and
e) adjusting the pH with one or more pH modifiers and finally adjusting the weight using purified water and mixing.

In yet another general aspect, the present invention provides for a method of reducing the microbial log count of Pseudomonas aeruginosa in a mouse superficial skin model with a thermal injury. The method includes topically administering the topical pharmaceutical composition described herein.

### DETAILED DESCRIPTION OF THE INVENTION

This invention relates to an improved pharmaceutical composition for topical application to treat burn wounds and infections, wherein the active drug is in the nanonized form.

The pharmaceutical composition for topical application may be in the form of cream, lotion, ointment or gel.

The pharmaceutical composition includes an antimicrobial drug and an antiseptic as the active ingredients. The antimicrobial drug as used herein is silver sulfadiazine in a concentration of 0.1%-1.0% w/w and the antiseptic is chlorhexidine gluconate in a concentration of 0.2% w/w. Silver sulfadiazine is dispersed and chlorhexidine gluconate is solubilized in a cream or lotion base, wherein silver sulfadiazine is in nanonized form.

The term “nanonized” as used herein refers to a particle size as expressed by Z-average or size average molar mass, of less than or equal to 600 nm, e.g., Z-average molar mass between 150-500 nm, determined using known particle size determination methods, e.g., using a Nanosizer or Zetasizer.

The Z-average is defined mathematically using the following formula:

$$Z \text{-} Average or Size Average Molar Mass = Mz = \frac{\sum {N}_{i} {M}_{i}^{3}}{\sum {N}_{i} {M}_{i}^{2}}$$

where Nᵢ is the number of molecules whose weight is and wherein the summation is from i=1 to i=∞.

Polydispersity Index (PI), an indicator of the spread in molar mass, is a parameter also available using a Nanosizer or Zetasizer. The PI value for silver sulfadiazine in the nanonized formulation is between 0.4-0.8, for example, 0.5-0.6.

Nanonization is a size reduction technique that leads to an increased particle surface area and thus increased dissolution velocity. Quicker absorption of fine sized particles having increased surface area leads to improved efficacy and correspondingly also a dose reduction. Also, in the present case, there is no significant increase in toxicity as a result of size reduction.

Topical compositions of silver sulfadiazine may be formulated using standard techniques known in the industry. For example, such formulations may be produced with a hydrophobic base with the addition of suitable viscosity enhancer.

Such hydrophobic bases may include mineral oils, such as liquid paraffin or a vegetable oil, such as peanut oil or castor oil.

Viscosity enhancers which may be used according to the characteristics of the base may include soft paraffin, aluminum stearate, cetostearyl alcohol, propylene glycol, polyethylene glycols, povidone, wool-fat, hydrogenated lanolin, beeswax, or mixtures thereof.

The present compositions may also be formulated into lotions or creams using methods known in the art. For example, such lotions may be formulated with an aqueous or oily base and may include one or more of the following: viscosity enhancers, emulsifying agents, fragrance/perfumes, preservatives, chelating agents, pH modifiers, antioxidants, and the like.

Suitable preservative may include one or more of methylparaben, propylparaben, benzyl alcohol, benzoic acid, sodium benzoate, chlorocresol, sorbic acid and its salt or phenylethyl alcohol.

Suitable emulsifying agent may include one or more of cetomacrogol, non-ethoxylated glyceryl monostearate, carbopols, cetearyl alcohol, sodium stearoyl lactylate, or lecithin.

Suitable chelating agents may include one or more of dimercaprol, ethylene diamine tetra acetic acid (EDTA), ethylene glycol tetra acetic acid, deferoxamine, or alfa lipoic acid.

Suitable pH modifiers may include citric acid, sodium citrate; acetic acid, sodium acetate; phosphoric acid, disodium orthophosphate; borax, sodium hydroxide, and the like.

Silver sulfadiazine is prone to oxidation, thereby causing the nanonized slurry to turn black on exposure to the environment during the milling process. This instability problem is more pronounced in case of the nanonized product (due to increased surface area) as compared to the already marketed micronized product. Addition of suitable antioxidants is useful in overcoming this instability problem.

The antioxidant may be hydrogen peroxide, sodium metabisulfite, butylated hydroxytoluene, butylated hydroxyanisole, propyl gallate, ethyl gallate, methyl gallate, ascorbic acid, tocopherol, or mixtures thereof.

According to one embodiment of the invention, the improved topical pharmaceutical composition contains a combination of butylated hydroxytoluene, hydrogen peroxide, and sodium metabisulfite, for use as antioxidants.

The present invention also relates to a method of preparation of the nanonized topical pharmaceutical composition of silver sulfadiazine. The process includes the steps of:
a) dispersing a viscosity enhancer and one or more pharmaceutically acceptable excipients in water to form a slurry;
b) adding and mixing silver sulfadiazine to the slurry of step a);
c) charging the slurry in a Dynomill and unloading after the particle size of silver sulfadiazine in the slurry is in the nano-range;
d) preparing the oily phase by mixing together one or more hydrophobic bases and heating the mixture to obtain a molten mass;
e) preparing the aqueous phase by heating purified water and dissolving a chelating agent and preservative in it;
f) slowly adding the oily phase of step d) to the aqueous phase of step e) and homogenizing to form an emulsion;
g) adding chlorhexidine gluconate to the cooled bulk of step f);
h) adding and mixing silver sulfadiazine slurry of step c) into the bulk of step g); and
i) adjusting the pH using one or more pH modifiers and finally adjusting the weight using purified water and mixing.

### Animal Model Studies

The improved efficacy of the nanonized pharmaceutical composition of the present invention has been demonstrated by in vivo efficacy studies in burn wound infection model by comparing the log reduction of Pseudomonas aeruginosa using various concentrations of nanonized silver sulfadiazine to an untreated control, commercially available Silverex® (1% micronised silver sulfadiazine) and levofloxacin therapy. Swiss albino mice were put into six groups and their backs were shaven. Thermal injury was induced in the shaven area by applying a heated brass rod on the back of the animals for 15 seconds to 20 seconds. For establishment of a topical infection, an inoculum containing approximately 2×10⁵ bacteria (Pseudomonas aeruginosa) was injected subcutaneously (100 μL) on the sites of the burn. One hour post infection, the mice were treated using 100 mg of cream formulations of different drug concentrations. In case of all the cream formulations, 100 mg cream was applied topically twice daily for 14 days. One of the groups of mice was given levofloxacin orally for 7 days at 25 mg/kg body weight as a reference standard.

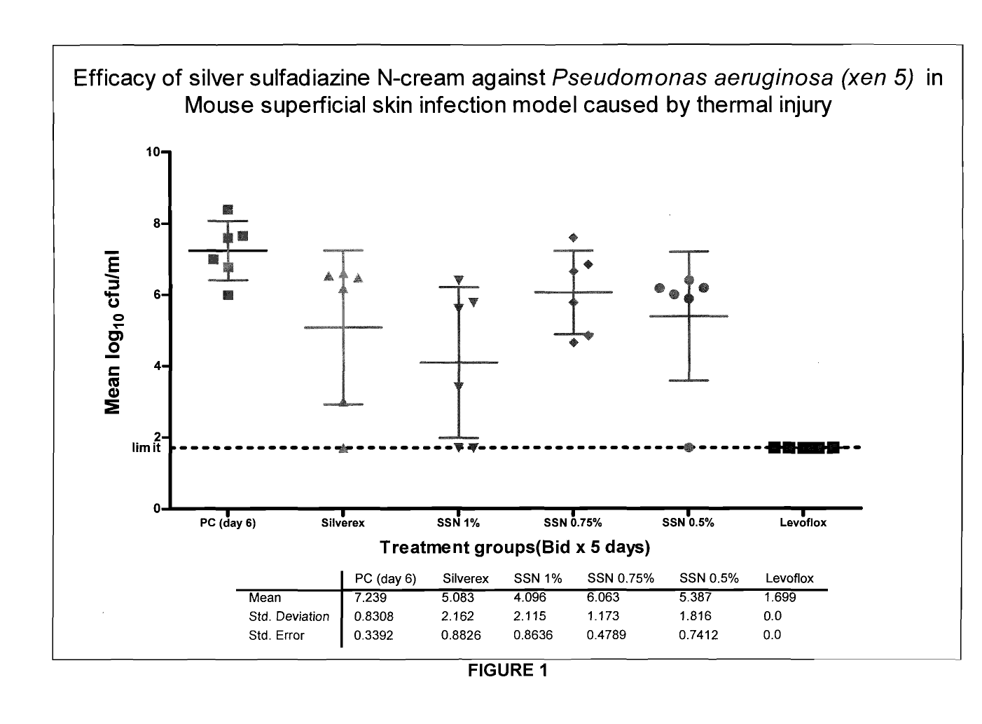

The results of the efficacy of silver sulfadiazine nanonized cream against Pseudomonas aeruginosa in mouse superficial skin model caused by thermal injury, on day 15 have been shown in FIG. 1 and Table 1.

TABLE 1
Log reduction of microbial count

| | |
|---|---|
| Silver sulfadiazine nanonized cream 0.5% | 1.85 log₁₀ |
| Silver sulfadiazine nanonized cream 0.75% | 1.17 log₁₀ |
| Silver sulfadiazine nanonized cream 1.0% | 3.14 log₁₀ |
| Silverex 1% | 2.15 log₁₀ |
| Levofloxacin (25 mg/kg BW) | 5.53 log₁₀ |

As seen from the results of the in vivo efficacy studies, 1% silver sulfadiazine nanonized (SSN) cream leads to greater log reduction in the Pseudomonas aeruginosa count in mouse superficial skin model caused by thermal injury as compared to the marketed product Silverex® 1%. While the microbial log reduction using 0.5% silver sulfadiazine nanonized cream is found to be similar to the marketed 1% Silverex®.

To further evaluate the efficacy of silver sulfadiazine (Nanonized) cream in the Thermal Injury Model in male Sprague Dawley rats after dermal application once daily for 15 consecutive days, histopathological evaluation of the tissue samples from the site of injury was carried out. The microscopic lesions were evaluated for necrosis, inflammatory cells, immature granulation tissue, mature granulation tissue, fibrosis and reepithelization to interpret the various wound healing events.

Marked to severe necrosis was observed in all the animals belonging to different treatment groups including the control, indicating the accuracy and precision of the thermal injury process. Hence, necrosis, in the present study is not a measure of a wound healing process. The animals were grouped in five sets, namely Group Ito Group V. Group I consisted of untreated control; Group II was treated with 0.5% nanonized silver sulfadiazine (SSN); Group III with 0.75% SSN; Group IV with 1% SSN; and Group V was treated with micronized Silverex 1%.

The mean score of inflammation (i.e., presence of inflammatory cells) was much less in Group II animals (0.5% SSN) compared with Group I (control) and other treatment Group III (0.75% SSN), Group IV (1% SSN) and Group V (1% Silverex). Similarly, the mean scores of granulation tissue (immature, mature and fibrosis) were low in Group II animals (0.5% SSN) compared with Group I (control) and other treatment Group III (0.75% SSN), Group IV (1% SSN) and Group V (1% Silverex).

The reepithelization (expressed as percent closure of wound by newly forming squamous cells) was high in Group II animals (0.5% SSN) followed by Group III (0.75% SSN), Group IV (1% SSN) and Group V (1% Silverex).

Tables 2, 3, & 4 present the mean histopathological scores of various wound healing parameters studied in the present study.

TABLE 2
Mean Histopathology Score of Healing Parameters

| | Wound Healing Parameters | | | | | |
|---|---|---|---|---|---|---|
| | | Inflammatory | Immature | Mature | | |
| Group | Necrosis | Cells | GT | GT | Fibrosis | Reepithelization |
| I | 4.4 | 2.9 | 2.1 | 1.4 | 1.1 | 22.5 |
| II | 4.6 | 1.3 | 1.7 | 0.9 | 1.1 | 41.0 |
| III | 4.8 | 1.7 | 1.8 | 0.8 | 1.4 | 28.5 |
| IV | 4.5 | 1.5 | 1.4 | 1.0 | 1.3 | 27.0 |
| V | 4.7 | 2.4 | 2.0 | 1.1 | 0.5 | 31.0 |

TABLE 3
Summary of Reepithelization (%) Scores

| | | Silver Sulfadiazine | | | |
|---|---|---|---|---|---|
| Reepithelization | | (Nanonized) | | | Silverex |
| Score | Placebo | 0.5% | 0.75% | 1% | 1% |
| ≦5% | 4 | 2 | 3 | — | 2 |
| ≦10% | 3 | 3 | 1 | 5 | 2 |
| ≦25% | 1 | 1 | 1 | 2 | 2 |
| ≦50% | — | — | 3 | 1 | 1 |
| ≦75% | 1 | — | 2 | 1 | 2 |
| ≦100% | 1 | 4 | — | 1 | 1 |

TABLE 4
Summary of Inflammatory Cells

| | | Silver Sulfadiazine | | | |
|---|---|---|---|---|---|
| Inflammatory Cells | | (Nanonized) | | | Silverex |
| Score | Placebo | 0.50% | 0.75% | 1% | 1% |
| 0.5 (very minimal) | — | 3 | — | — | — |
| 1 (minimal) | — | 2 | 4 | 5 | 6 |
| 2 (mild) | 4 | 5 | 5 | 5 | 4 |
| 3 (moderate) | 3 | — | 1 | — | — |
| 4 (marked) | 3 | — | — | — | — |

Overall, all the healing parameters, viz., inflammation, granulation tissue and reepithelization evaluated in the present study showed improved results in animal groups treated with nanonized silver sulfadiazine formulation when compared with the untreated control group and the group treated with 1% Silverex.

### Pharmacokinetic Studies

The plasma exposures of silver and sulfadiazine were determined on day 15 following once daily dermal application of different strengths of silver sulfadiazine (Nanonized) cream as Test Items (SSN 0.5%, 0.75% and 1%) and Silverex cream 1% (as reference item). Sulfadiazine was quantitated by the LC-MS/MS method. The mouse plasma samples were analyzed by API 4000 QTRAP MS/MS detector in positive ion mode using a Kromasil C18, (100×4.6 mm), 3.5μ column. The mobile phase used was acetonitrile:water:formic Acid (80:20:0.05) at a flow rate of 0.6 ml/min.

Silver concentrations were estimated using ICPMS instrument (Inductively Coupled Plasma—Mass Spectrometry). The conditions for ICPMS employed were—silver with 107 amu, an injection volume of about 1 ml, with the carrier gas flow rate of 0.95 l/min; make-up gas flow rate of 0.2 l/min; and plasma gas flow rate of 15 l/min. The readings were taken in replicates of three.

The pharmacokinetic parameters for test and reference items are given in Table 5.

TABLE 5
Pharmacokinetic Parameter Values for Silver and Sulfadiazine

| | |
|---|---|
| Cₘₐₓ (μg/ml) | AUC₀₋ₜ (h * μg/ml) |

| Formulation | Silver | Sulfadiazine | Silver | Sulfadiazine |
|---|---|---|---|---|
| Silverex-1% | 0.22 | 27.63 | 3.88 | 493.56 |
| SSN-0.5% | 0.14 | 16.71 | 2.44 | 254.47 |
| SSN-0.75% | 0.14 | 19.99 | 3.02 | 361.74 |
| SSN-1% | 0.21 | 34.52 | 4.68 | 541.95 |

The Cₘₐₓ of sulfadiazine for different SSN formulation prototypes increased from 16.71 μg/mL to 34.52 μg/mL with the increase in strengths from 0.5% to 1%. Similarly, the AUC increased from 254.47 h*μg/mL to 541.95 h*μg/mL with the increase in SSN strengths. The AUC ratio (Test/Reference) ranged from 0.52 to 1.10 after 15 days of dermal application.

The AUC of silver was calculated from silver concentrations of pooled plasma samples. The exposure of silver also increased from 2.44 h*μg/mL to 4.68 h*μg/mL with the increase in strengths of SSN creams from 0.5% to 1%. The AUC ratio (Test/Reference) ranged from 0.63 to 1.21 after 15 days of dermal application.

Both silver and sulfadiazine were found to be absorbed from the topical application of silver sulfadiazine creams in rat. The systemic exposure of both the components (silver and sulfadiazine) was comparable from 1% test and reference product and dose dependent for SSN after topical application for 15 days.

The silver nanoparticles with their large surface area and reduced particle size provide improved contact and penetration with bacterial cell, which together enhances its antimicrobial activity. The anti-inflammatory effects of silver sulfadiazine depend on concentration of silver at the site, release of silver from the carrier and species of silver used.

The invention is further illustrated by the following non-limiting examples.

### EXAMPLES

### Silver Sulfadiazine (Nanonized Cream)

| | Qty (% w/w) | | | | |
|---|---|---|---|---|---|
| Ingredients | Example 1 | Example 2 | Example 3 | Example 4 | Example 5* |
| Part A: Silver Sulfadiazine | | | | | |
| Slurry (Nanonized) | | | | | |
| Silver Sulfadiazine USP | 0.10 | 0.25 | 0.50 | 0.75 | 1.00 |
| Hydrogen Peroxide Solution | 0.02 | 0.05 | 0.10 | 0.15 | 0.20 |
| Polyvinyl Pyrrolidone | 0.10 | 0.10 | 0.10 | 0.10 | 0.10 |
| Sodium Metabisulphite | 0.05 | 0.05 | 0.05 | 0.05 | 0.05 |
| Purified Water | 40.00 | 40.00 | 40.00 | 40.00 | 40.00 |
| Part B: Drug | | | | | |
| Chlorhexidine Gluconate | 0.20 | 0.20 | 0.20 | 0.20 | 0.20 |
| Solution IP eq to | | | | | |
| Chlorhexidine | | | | | |
| Part C: Oil Phase | | | | | |
| Cetostearyl Alcohol | 9.60 | 9.60 | 9.60 | 9.60 | 9.60 |
| Cetomacrogol 1000 | 2.50 | 2.50 | 2.50 | 2.50 | 2.50 |
| Light Liquid Paraffin | 8.00 | 8.00 | 8.00 | 8.00 | 8.00 |
| Butylated Hydroxytoluene | 0.10 | 0.10 | 0.10 | 0.10 | 0.10 |
| Part D: Water Phase | | | | | |
| Purified Water | 30.00 | 30.00 | 30.00 | 30.00 | 30.00 |
| Disodium Edetate | 0.01 | 0.01 | 0.01 | 0.01 | 0.01 |
| Chlorocresol | 0.10 | 0.10 | 0.10 | 0.10 | 0.10 |
| Part E | | | | | |
| Phosphoric Acid | qs | qs | qs | qs | qs |
| Disodium Orthophosphate | qs | qs | qs | qs | qs |
| Purified Water | qs to 100 g | qs to 100 g | qs to 100 g | qs to 100 g | qs to 100 g |

*Particle size of Silver sulfadiazine in the formulation was determined using Zetasizer with water as dispersant, and the Z-average or size average molar mass was found to be 465 nm.

### Brief Manufacturing Procedure

### Preparation of Part A

1. Hydrogen peroxide and sodium metabisulphite were dissolved in purified water.
2. Polyvinyl pyrrolidone was dispersed into the bulk of step 1.
3. Silver sulfadiazine was mixed into it.
4. The slurry was passed through the Dynomill and collected and the Dynomill was rinsed with purified water.
5. The slurry and rinsing were used in the fabrication of the cream.

### Preparation of Cream

1. The oil phase was prepared by mixing together cetostearyl alcohol, Cetomacrogol 1000, light liquid paraffin, and butylated hydroxytoluene and heating to 65° C. to 70° C.
2. The water phase was prepared by heating purified water to 65° C. to 70° C. and dissolving disodium edetate and chlorocresol in it.
3. The oil phase was added slowly under homogenization to the water phase to form an emulsion, which was then cooled under stirring.
4. Chlorhexidine gluconate was added and mixed into the bulk of step 3.
5. Silver sulfadiazine slurry and the rinsings were added and mixed into the bulk of step 4.
6. The pH was adjusted using orthophosphoric acid and disodium orthophosphate.
7. Final weight adjustment was done using purified water followed by mixing.

| | Qty (% w/w) | |
|---|---|---|
| Ingredients | Example 6 | Example 7 |
| Part A: Silver Sulfadiazine Slurry | | |
| (Nanonized) | | |
| Silver Sulfadiazine USP | 1.00 | 1.00 |
| Hydrogen Peroxide Solution | — | 0.10 |
| Polyvinyl Pyrrolidone | 0.10 | 0.10 |
| Sodium Metabisulphite | — | — |
| Purified Water | 40.0 | 40.00 |
| Part B: Drug | | |
| Chlorhexidine Gluconate Solution IP eq to | 0.20 | 0.20 |
| Chlorhexidine | | |
| Part C: Oil Phase | | |
| Cetostearyl Alcohol | 9.60 | 9.60 |
| Cetomacrogol 1000 | 2.50 | 2.50 |
| Light Liquid Paraffin | 8.00 | 8.00 |
| Butylated Hydroxytoluene | 0.10 | 0.10 |
| Part D: Water Phase | | |
| Purified Water | 30.00 | 30.00 |
| Disodium Edetate | 0.01 | 0.01 |
| Chlorocresol | 0.10 | 0.10 |
| Part E | | |
| Phosphoric Acid | qs | qs |
| Disodium Orthophosphate | qs | qs |
| Purified Water | qs to 100 g | qs to 100 g |

Formulations 6 & 7 were prepared in the same manner as described above, except omitting the step of addition of hydrogen peroxide and sodium metabisulphite in Example 6 and excluding sodium metabisulphite addition in Example 7.

The formulations of Examples 5, 6 & 7 were charged on stability to observe the effect of various antioxidants on product stability. The results of the stability studies are compiled below:

| S No. | Formulation | Observations on Stability |
|---|---|---|
| 1. | Example 5 | No color change observed on stability |
| 2. | Example 6 | SS slurry became black immediately |
| 3. | Example 7 | Samples became black on stability |

From the above results, it is evident that a combination of hydrogen peroxide (50% w/w) and sodium metabisulfite is effective in preventing any color change of silver sulfadiazine in the pharmaceutical composition.

1. A topical pharmaceutical composition comprising 0.1% w/w to 1% w/w silver sulfadiazine, 0.2% w/w chlorhexidine gluconate, and one or more pharmaceutically acceptable excipients; wherein the silver sulfadiazine has a Z-average molar mass between 150 nm to 500 nm.

2. The topical pharmaceutical composition according to claim 1, wherein the pharmaceutically acceptable excipients comprise one or more of thickening agents, emulsifying agents, fragrance/perfumes, preservatives, chelating agents, pH modifiers, and antioxidants.

3. The topical pharmaceutical composition according to claim 2, wherein the thickening agents comprises one or more of soft paraffin, aluminum stearate, cetostearyl alcohol, propylene glycol, polyethylene glycols, povidone, wool-fat, hydrogenated lanolin and beeswax.

4. The topical pharmaceutical composition according to claim 2, wherein the emulsifying agent comprises one or more of cetomacrogol, non-ethoxylated glyceryl monostearate, carbopols, cetearyl alcohol, sodium stearoyl lactylate and lecithin.

5. The topical pharmaceutical composition according to claim 2, wherein the preservative comprises one or more of methylparaben, propylparaben, benzyl alcohol, benzoic acid, sodium benzoate, chlorocresol, sorbic acid and its salt, and phenylethyl alcohol.

6. The topical pharmaceutical composition according to claim 2, wherein the chelating agent comprises one or more of dimercaprol, ethylene diamine tetra acetic acid (EDTA), ethylene glycol tetra acetic acid, deferoxamine and alfa lipoic acid.

7. The topical pharmaceutical composition according to claim 2, wherein the pH modifier comprises one or more of citric acid, sodium citrate; acetic acid, sodium acetate; phosphoric acid, disodium orthophosphate; borax and sodium hydroxide.

8. The topical pharmaceutical composition according to claim 2, wherein the antioxidant comprises one or more of hydrogen peroxide, sodium metabisulfite, butylated hydroxytoluene, butylated hydroxyanisole, propyl gallate, ethyl gallate, methyl gallate, ascorbic acid, or tocopherol.

9. The topical pharmaceutical composition according to claim 8, wherein the antioxidant comprises a combination of hydrogen peroxide, sodium metabisulfite, and butylated hydroxytoluene.

10. The topical pharmaceutical composition according to claim 1, wherein the composition is a cream, lotion, ointment or gel.

11. A process for the preparation of a topical pharmaceutical composition of claim 2, wherein the process comprises the steps of:
a) dispersing the silver sulfadiazine and the thickening agent(s) in water;
b) charging the dispersion of step a) in a Dynomill and unloading the slurry when silver sulfadiazine attains a Z-average particle size between 150 nm to 500 nm;
c) emulsifying suitable oil and aqueous phases containing excipients;
d) adding chlorhexidine gluconate and the slurry containing nanonized silver sulfadiazine to the emulsion of step c); and
e) adjusting the pH with one or more pH modifiers and finally adjusting the weight using purified water and mixing.

12. (canceled)

13. The topical pharmaceutical composition according to claim 1, whereby said topical pharmaceutical composition exhibits a microbial log reduction of Pseudomonas aeruginosa count in a mouse superficial skin model with thermal injury that is greater than or equal to that achieved by Silverex®.
